(19) United States
(12) Patent Application Publication
(10) Pub. No.: US 2014/0228299 A1
(43) Pub. Date: Aug. 14, 2014

(54) PROPHYLACTIC AND THERAPEUTIC AGENT FOR NEUROLOGICAL DISEASES USING LIPOPROTEINS AND PROPHYLACTIC AND THERAPEUTIC METHOD FOR NEUROLOGICAL DISEASES

(71) Applicants: National University Corporation Tokyo Medical and Dental University, Tokyo (JP); National University Corporation Kumamoto University, Kumamoto (JP)

(72) Inventor: Hideki Hayashi, Kumamoto-shi (JP)

(73) Assignees: National University Corporation Tokyo Medical and Dental University, Tokyo (JP); National University Corporation Kumamoto University, Kumamoto (JP)

(21) Appl. No.: 14/346,002

(22) PCT Filed: Sep. 19, 2012
(86) PCT No.: PCT/JP2012/073920

§ 371 Date: Mar. 20, 2014

(30) Foreign Application Priority Data
Sep. 20, 2011 (JP) 2011-205390
May 25, 2012 (JP) 2012-119680

(51) Int. Cl. A61K 38/17

(52) U.S. Cl. CPC A61K 38/1709
USPC 514/17.7

(57) ABSTRACT

The present invention provides an agent for neurological disease, the agent comprising lipoprotein containing apolipoprotein E as the active substance thereof, in which a neuroprotective system via activation of neuroprotective molecules and/or the inactivation of neurodegeneration-inducing molecules, mediated by lipoprotein receptors, may work as a mechanism, and a prophylactic and therapeutic method for neurological diseases. The lipoproteins containing apolipoprotein E and/or neuroprotective system through, as the action mechanism, the activation of neuroprotective molecules and the inactivation of neurodegeneration-inducing molecules that are mediated by lipoprotein receptors have prophylactic and therapeutic effects of, including, inhibiting nerve cell apoptosis, on neurological disease such as various neurodegenerative diseases accompanied by nerve cell apoptosis as the essential condition. The invention provides the agent comprising apolipoprotein E-containing lipoproteins and/or having an action mechanism through the activation of neuroprotective molecules and the inactivation of neurodegeneration-inducing molecules that are mediated by lipoprotein receptors.

### CROSS REFERENCE TO RELATED APPLICATIONS

This application relates to and claims priority from PCT App. No. PCT/JP2012/073920 filed Sep. 19, 2012, the entire contents of which are incorporated herein fully by reference, and which in turn claims priority from JP Ser. No. JP2012-119680 filed May 25, 2012 and JP2011-205390 filed Sep. 20, 2011.

### FIGURE SELECTED FOR PUBLICATION

FIG. 1

### BACKGROUND OF THE INVENTION

1. Field of the Invention

The present invention relates to an inhibitor of the neuroprotective inhibitory effect of α2-macroglobulin and an ophthalmologic composition thereof utilizing lipoprotein.

2. Description of the Related Art

Intractable neurological diseases such as Alzheimer's disease, Parkinson's disease, amyotrophic lateral sclerosis and spinocerebellar degeneration, developing more frequently in people of the middle and old age, are considered as neurodegenerative diseases having a common onset mechanism because any such diseases accumulate abnormal proteins inside the nerve cells leading to an occurrence of selective cell death of the nerve cells. Therefore, such neurodegenerative diseases are considered as probably causing abnormality in the apoptosis control mechanism of normal cells.

A degenerative disease of the retina or the optic nerve is one of various neurodegenerative diseases in which an apoptosis of the nerve cells or the like is an inevitable cause. In Japan, degenerative diseases of the retina and the optic nerve are almost all causes of adventitious blindness. In the progressive super aging society like in Japan, prevention of the blindness likely caused by the optic neurodegenerative diseases, such as so-to-speak “an adult disease of the eye” like glaucoma and diabetic retinopathy is an urgent and serious concern to be solved. The glaucoma is the progressive neurodegenerative disease along with degenerating and omitting the nerve cells, and it is the disease that is ranked first in Japan and second in the world as the disease causing the blindness. The sense of sight is one of the most important senses and, even if the eyesight would not be lost completely, quality of life due to visual impairments will be remarkably deteriorated. Further, the diabetic retinopathy, a major disease causing blindness in the generation of working-age in the advanced countries, is known as one of the three major complications of diabetes. From these backgrounds, development of a new treatment method is an urgent need for both diseases.

As described above, the diabetic retinopathy and glaucoma are known that the retinal ganglion cells structuring the optic nerve cells undergo damages, however, in each disease, not only details of its onset mechanism is not yet made clear but also it is extremely difficult to sustain the visual function because the treatment is difficult once it would have developed. This is also a big social issue from the standpoint of quality of life.

Recent years, it has been becoming clear that the glia cells which support the nerve cells physically and physiologically are involved positively in a variety of nervous functions. Moreover, it has been found that lipoproteins secreted from the glia cells play an important role in the lipid metabolism in the central nervous system (Non-Patent Literature Document 1).

Therefore, in order to elucidate mechanisms of optic neurodegeneration in optic neurological diseases and develop a method for treatment thereof, the inventors of the present invention have made extensive studies on the control over cell death of the retinal nerve cells in the optic neurodegeneration of such as glaucoma and retinal retinopathy focusing on glia-cell-derived lipoproteins as a factor protecting the retinal neurons. Specifically, if the glia-cell-derived lipoprotein could inhibit cell death of the optic nerve cells on those optic neurodegenerative diseases, it is expected to be able to result in elucidation of the mechanisms of cell death of the optic nerve cells on the neurodegeneration of other neurodegenerative diseases and in development of a treatment method for the neurodegeneration.

As a result, the inventors of the present invention have confirmed that a lipoprotein containing a glia-cell-derived apolipoprotein E showed a neuroprotective effect on apoptosis (Non-Patent Literature Document 2). The inventors of the present invention have confirmed that a lipoprotein containing an apolipoprotein E (E-LP) produced by the glia cells of the central nervous system could inhibit not only apoptosis induced by the omission of a nutrient for rat retinal ganglion neurons, but also the calcineurin activation involved in a pathway of the neuronal cell death and showed the protective action against the neuronal cell death by oxidation stress. (Non-Patent Literature Document 3)

As described above, the inventors of the present invention have confirmed by experiments using an in vitro system that the lipoprotein containing apolipoprotein E had the neuroprotective effects on apoptosis. As a large amount of lipoproteins are essentially present in human body, however, it is likely unclear actually whether the neuroprotective effects on the apoptosis is based on administered lipoprotein containing apolipoprotein E or not, when the lipoprotein containing apolipoprotein E are administered to human. Thus, the inventors of the present invention tried to confirm whether the lipoprotein containing apolipoprotein E had the neuroprotective effects on the apoptosis in an in vivo system by injecting the lipoprotein containing apolipoprotein E into the mouse's vitreous body in which a large amount of lipoproteins are present. As a result, the inventors of the present invention have found that the lipoprotein containing apolipoprotein E had the neuroprotective effects on apoptosis in such in vivo system, as well. Moreover, it has been found that a complex of the lipoprotein containing apolipoprotein E along with a neuroprotective molecule such as LRP1 or the like had likewise the neuroprotective effects on apoptosis in an in vivo system as well. Therefore, the present invention has been completed on the basis of the findings that a neuroprotective system having neuroprotective effects on apoptosis in an in vivo system works as well, in which the neuroprotective molecule was activated and/or the neurodegeneration-inducing molecule was inactivated by a lipoprotein containing apolipoprotein E and/or a lipoprotein receptor.

### PRIOR ARTS

### Non-Patent Literature Documents

[Non-Patent Literature Document 1] Hayashi, H., et al., J. Neurosci., Feb. 21, 2007, 27(8): 1933-1941
[Non-Patent Literature Document 2] Hayashi, H., et al: Neurochemistry, Vol. 49, No. 2/3 Page 483 (2010)
[Non-Patent Literature Document 3] Hayashi, H., et al., J Biol Chem: Vol. 284 No. 43 pp. 29605-29613(2009)

### ASPECTS AND SUMMARY OF THE INVENTION

### Problems to be Solved by the Invention

Following, one object of the present invention is to provide an inhibitor of the neuroprotective inhibitory effect of α2-macroglobulin and an ophthalmologic composition thereof utilizing lipoprotein.

The term “lipoprotein containing apolipoprotein E” as used herein is intended to mean the case in which apolipoprotein E and a lipoprotein are present together in the same medium, in addition to the case in which the apolipoprotein E is chemically associated with the lipoprotein.

The term “neuroprotective molecule” as used herein is intended to mean a molecule activated by the lipoprotein containing apolipoprotein E that demonstrates an neuroprotective effect may include, for example, but not limited to a lipoprotein receptor, phospholipase C, and protein kinase U. Furthermore, a molecule, so-called “neurodegeneration-inducing molecule”, inactivated (inhibited) thereby demonstrates reverse effects on neuroprotection may include, for example, but not limited to “neurodegeneration-inducing molecule”, NMDA receptor, calcium, and GSK3β.

Further, the term “lipoprotein receptor” as used herein is intended to mean a receptor of a lipoprotein receptor family which is connected to the apolipoprotein E in the central nervous system and which may include, for example, but not limited to LRP1 receptor, LDL receptor, ApoER2, receptor, VLDL receptor, LR11 receptor, LRP4 receptor, LRP1B receptor, Megalin receptor, LRP5 receptor, and LRP6.

Another object of the present invention is to provide a neurodegenerative inhibitor such as an apoptosis inhibitor containing a lipoprotein containing an apolipoprotein E, a lipoprotein receptor, a neuroprotective factor and a neurodegeneration-inducing factor as an active substance, for example, a prophylactic and therapeutic agent and so forth for such as glaucoma and diabetic retinopathy and a prophylactic and therapeutic method for neurological diseases using the prophylactic and therapeutic agent for neurological diseases containing such as a lipoprotein containing apolipoprotein E, a lipoprotein receptor, a neuroprotective factor, a neurodegeneration-inducing factor, or a neurodegenerative inhibitor and so forth.

Another object of the present invention as another example, is to provide a prophylactic and therapeutic agent for neurological diseases and a prophylactic and therapeutic method for neurological diseases, which can be used in an in vivo system, a neuroprotective system through a mediation of utilizing the lipoprotein containing apolipoprotein E or the lipoprotein receptor, the neuroprotective factor or the neurodegeneration-inducing factor and so forth.

### Means to Solve the Problems

In order to achieve the above objects, the present invention provides an inhibitor of the neuroprotective inhibitory effect of α2-macroglobulin containing a lipoprotein containing apolipoprotein E to inhibit the neuroprotective inhibitory effects of α2-macroglobulin by administration onto the eyeball by eyedrops or injections into the eyeball.

The present invention also provides an ophthalmologic composition characterized in that the composition is a composition comprising an lipoprotein containing apolipoprotein E and it inhibits the activity of a glutamic acid receptor of retinal ganglion cells present in the eyeball by the lipoprotein containing apolipoprotein E by administering the composition through eyedrops onto or injections into the eyeball, and it inhibits the neuroprotective inhibitory effects of α2-macroglobulin.

The present invention as a further mode provides a use of the ophthalmologic composition as a prophylactic and therapeutic agent for diabetic retinopathy.

In the present invention, the lipoprotein containing apolipoprotein E has the neuroprotective effects on apoptosis not only in an in vitro system but also in an in vivo system. Therefore, the present invention is expected to contribute to development of a prophylactic and therapeutic agent for glaucoma and so forth because it can inhibit the neurodegeneration in neurodegenerative diseases such as apoptosis of optic nerve cells. Moreover, it is expected to contribute to development of a prophylactic and therapeutic agent for Alzheimer's disease, Parkinson's disease and so forth because it has the neuroprotective effects on not only the optic nerve cells but also on the cerebral cortical neurons.

### BRIEF DESCRIPTION OF THE DRAWINGS

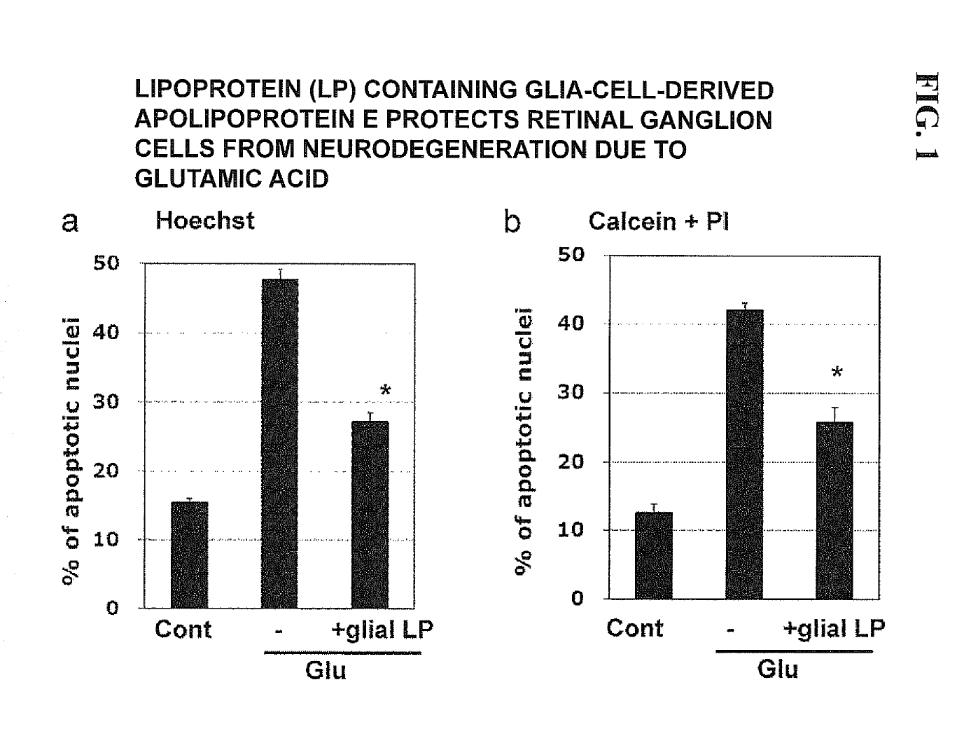

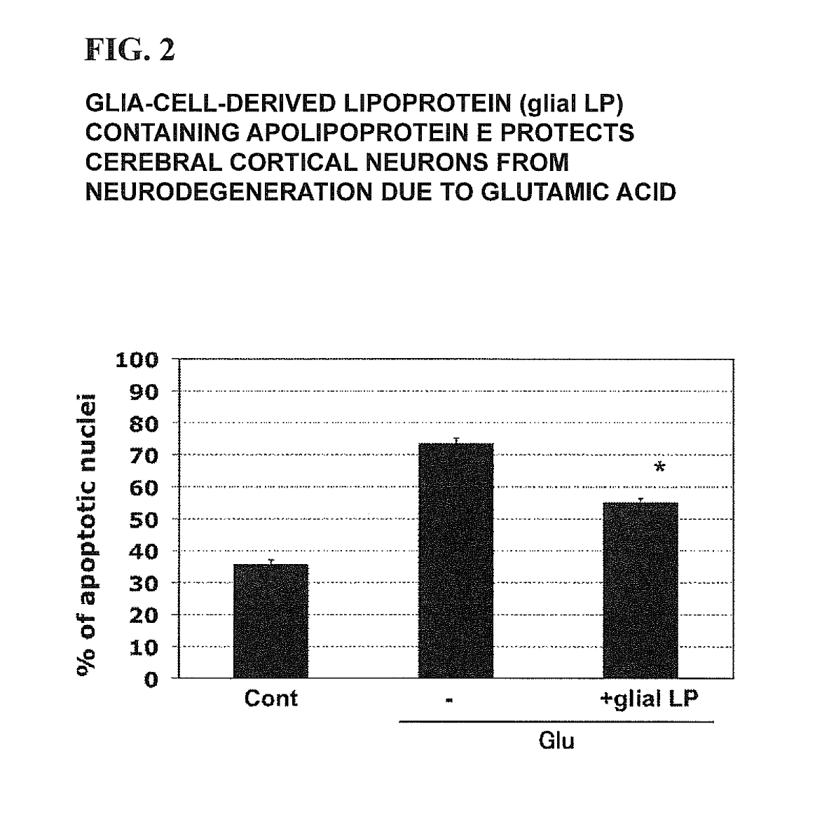

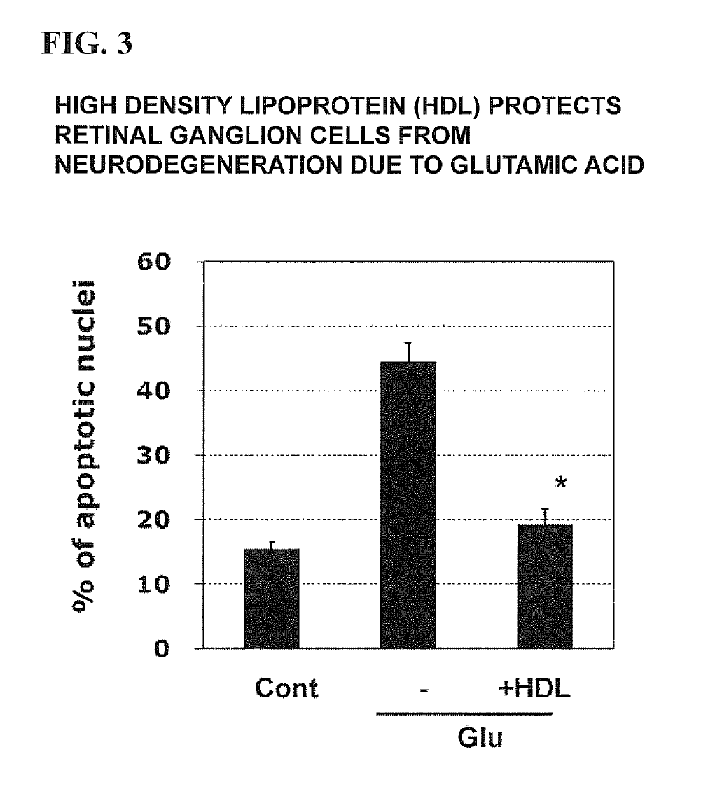

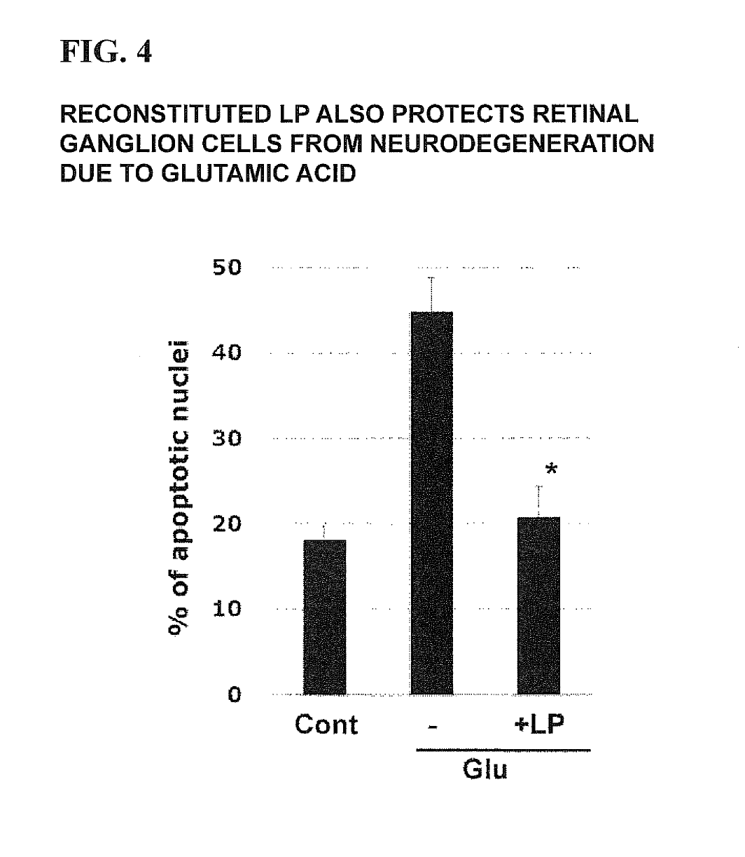

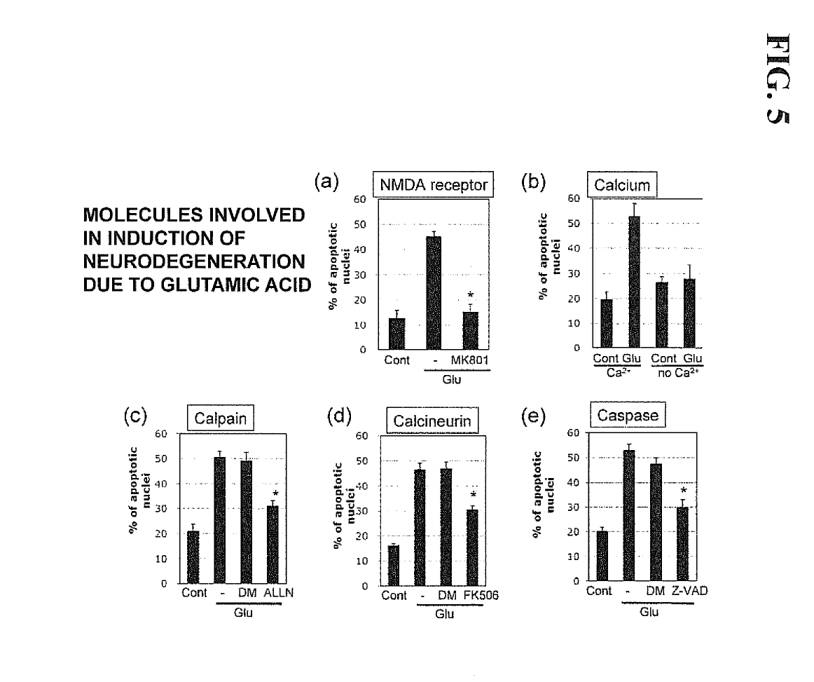

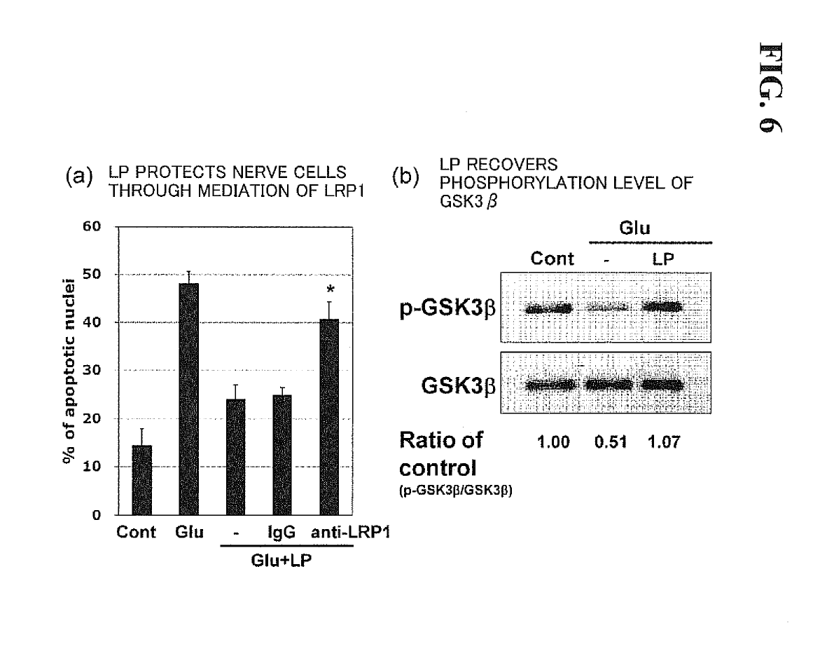

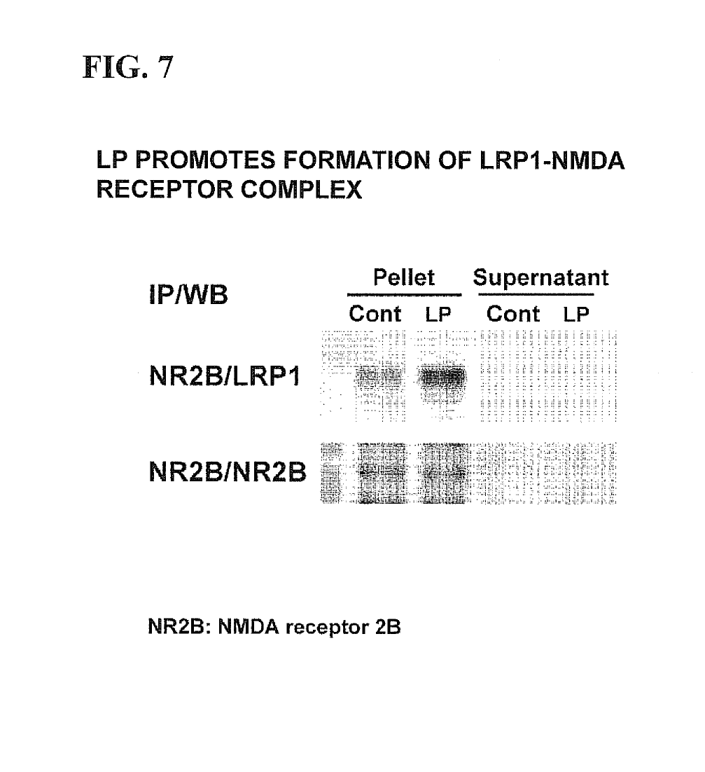

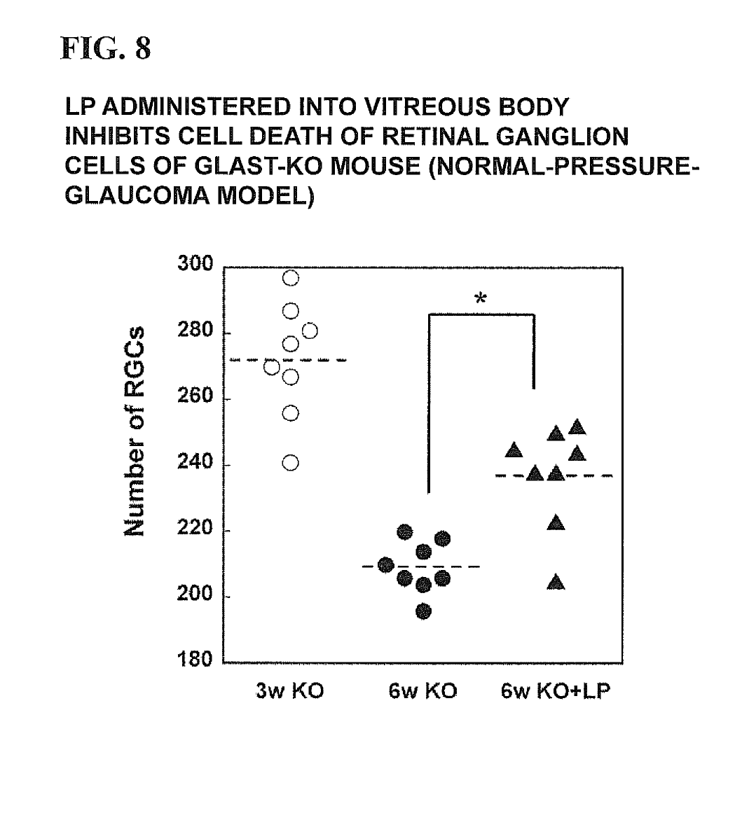

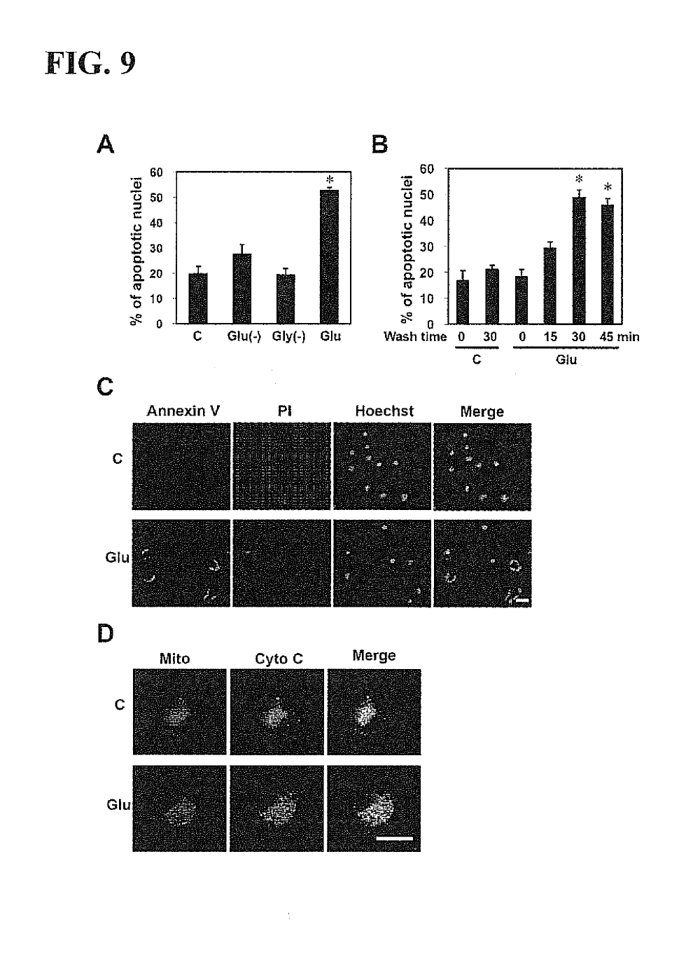

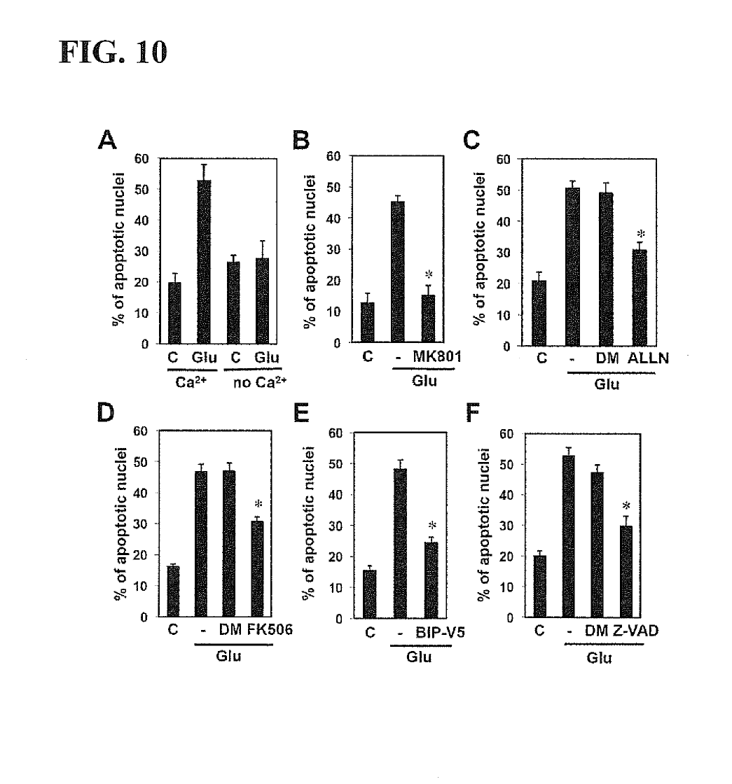

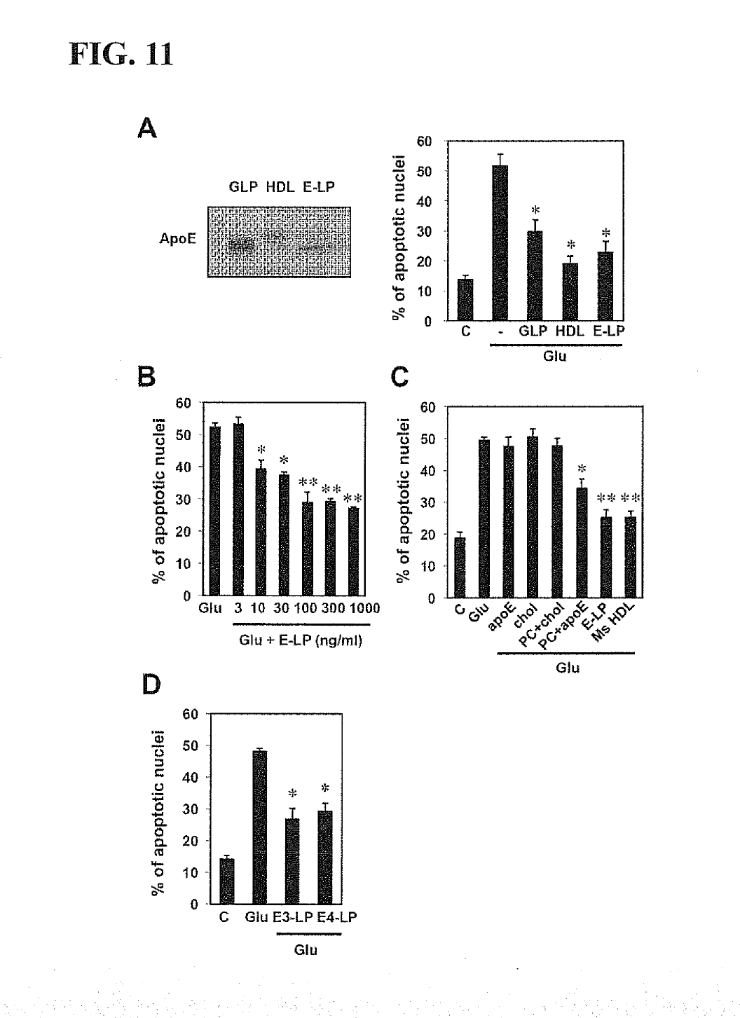

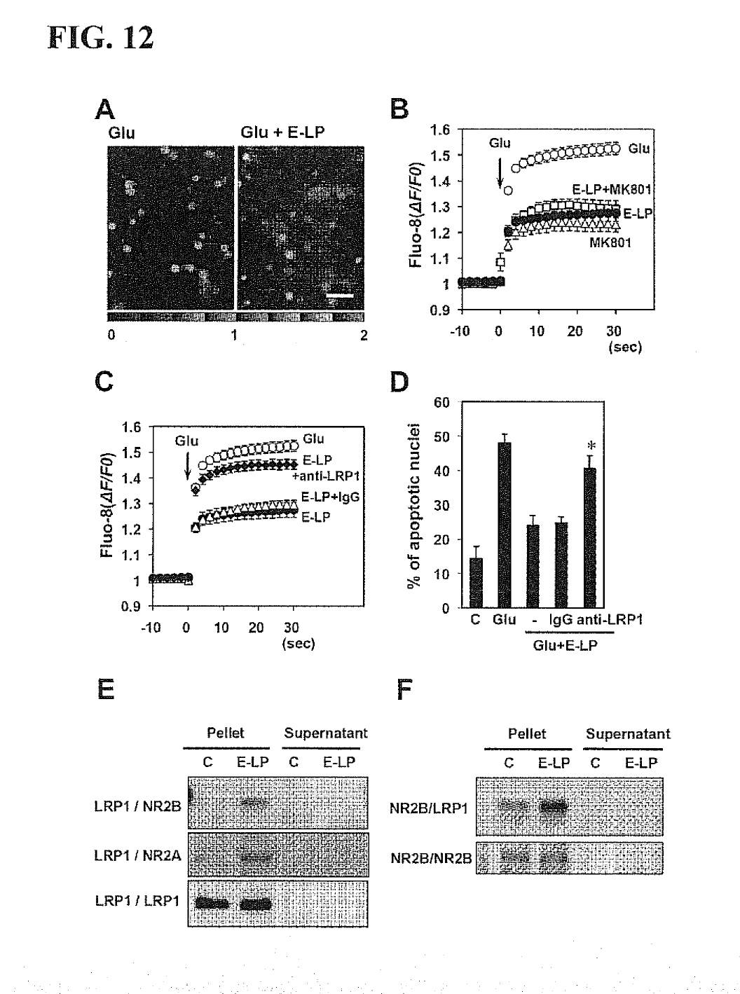

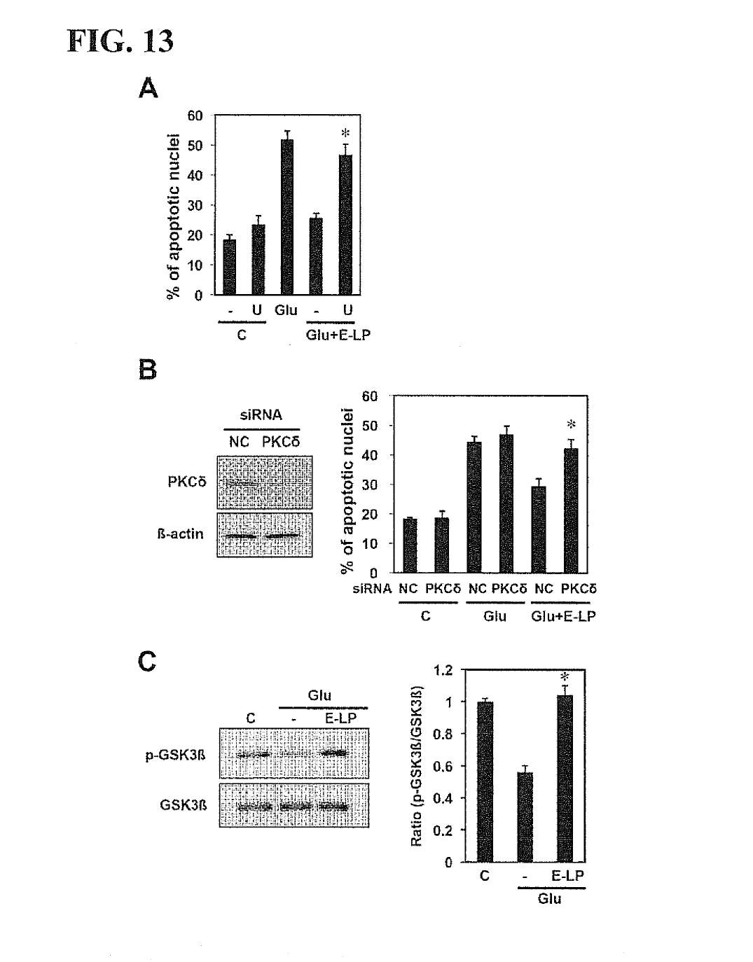

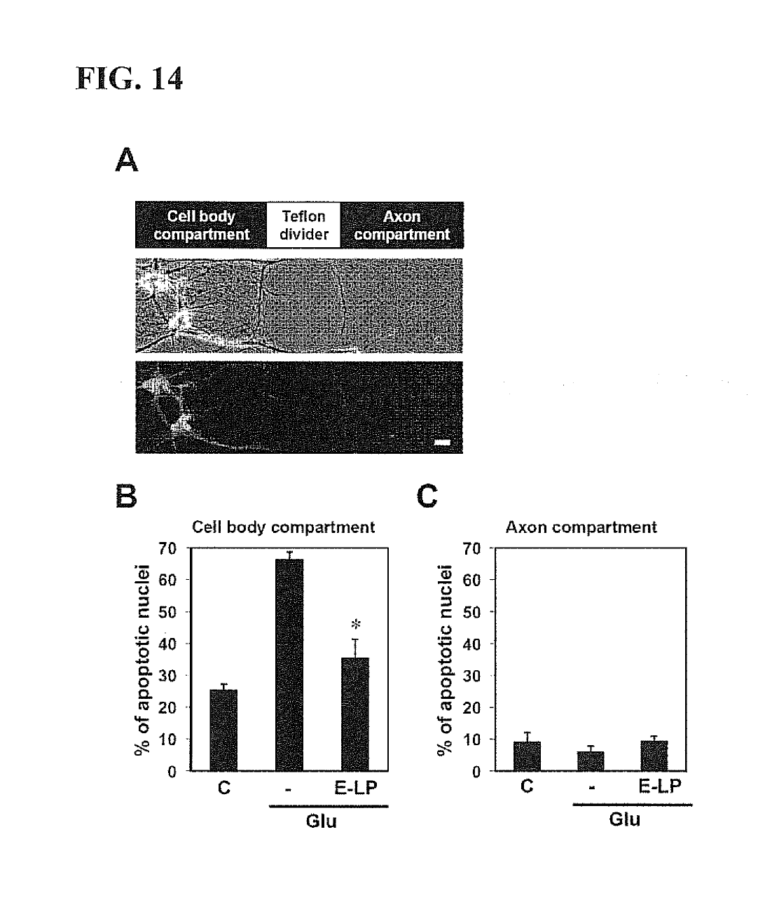

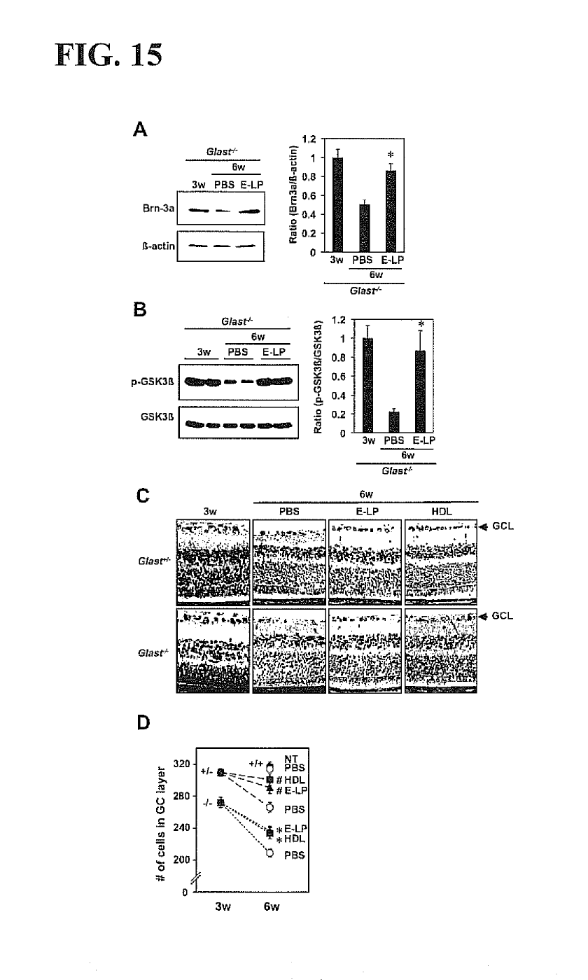

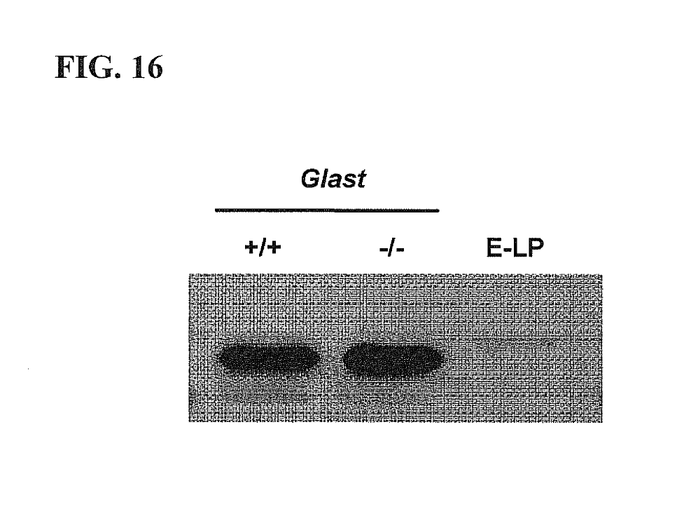

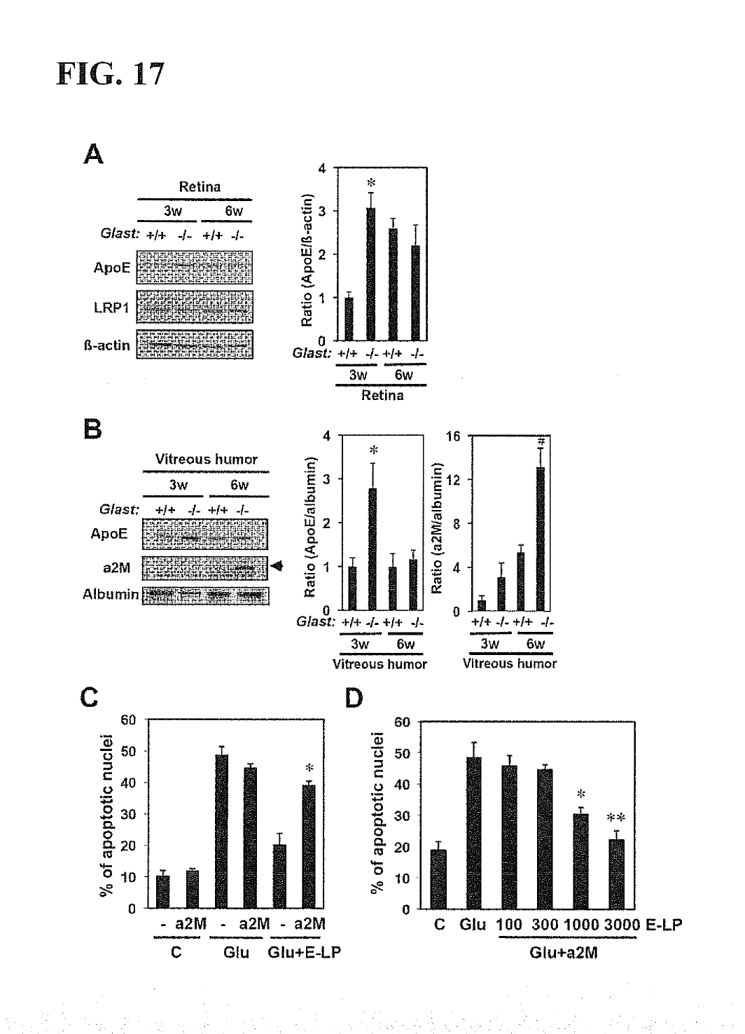

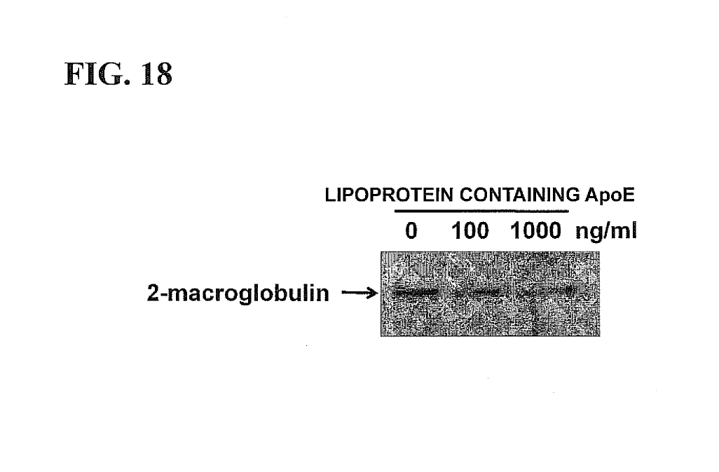

FIG. 1 A figure showing the protective effect of the retinal ganglion cells from neurodegeneration due to glutamic acid using glia-cell-derived lipoprotein (LP) containing apolipoprotein E (Example 3).

FIG. 2 A figure showing the protective effect of the cerebral cortical neurons from neurodegeneration due to glutamic acid using glia-cell-derived lipoprotein (LP) containing apolipoprotein E (Example 4).

FIG. 3 A figure showing the protective effect of the retinal ganglion cells from neurodegeneration due to glutamic acid using high density lipoprotein (HDL) (Example 6),

FIG. 4 A figure showing the protective effect of the retinal ganglion cells from neurodegeneration due to glutamic acid using reconstituted LP (Example 8).

FIG. 5 A figure showing the involvement of the following neurodegeneration-inducing molecules with induction of neurodegeneration due to glutamic acid (Example 9): (a) NMDA receptor; (b) calcium; (c) calpain; (d) calcineurin; and (e) caspase.

FIG. 6 (a) A figure showing the neuroprotective effect via LRP1 using lipoprotein containing apolipoprotein E. (b) A figure showing the effect on the recovery of GSK3b phosphorylation levels using the lipoprotein containing apolipoprotein E (Example 10).

FIG. 7 A figure showing the effect of lipoprotein containing apolipoprotein E on the promotion of formation of LRP1-NMDA receptor (Example 11).

FIG. 8 A figure showing the effect of the lipoprotein containing apolipoprotein E administered into the vitreous body on the inhibition of death of the retinal ganglion cells of a GLAST-KO mouse (a model mouse of normal-pressure-glaucoma) (Example 12).

FIG. 9 A figure showing the induction of apoptosis into RGC by a mixture of glutamic acid with glycine (Example 19).

FIG. 10 A figure showing a substance involving in the glutamic acid-induced neurotoxicity in RGC (Example 20).

FIG. 11 A figure showing a lipoprotein preventing the glutamic acid-induced apoptosis (Example 21).

FIG. 12 A figure showing the event that E-LP inhibits an increase in intracellular Ca²⁺ to be caused by an interaction of LRP1 with NMDA receptor (Example 22).

FIG. 13 A figure showing the protective effect of E-LP on the glutamic acid-induced neurotoxicity by phospholipase C, protein kinase Cδ and GSK3β (Example 23).

FIG. 14 A figure showing the glutamic acid-induced apoptosis in the cell body (Example 2e).

FIG. 15 A figure showing the recovery of the RGC existence in Glast+/− and Glast−/−mice by E-LP (Example 25).

FIG. 16 A figure showing the comparative plot results of the amount of the apolipoprotein E present in the vitreous bodies of the Glast+/+ and Glast−/− mice with the amount of the apolipoprotein E contained in E-LP injected into the vitreous bodies thereof (Example 26).

FIG. 17 A figure showing the inhibition of the inhibitory effect on the neuroprotection of α2 macroglobulin by E-LP (Example 27).

FIG. 18 A figure showing a decrease in an amount of α2 macroglobulin released from the glia cells (Example 28).

### DETAILED DESCRIPTION OF THE PREFERRED EMBODIMENTS

As described above, the inventors of the present invention have confirmed that the lipoprotein containing apolipoprotein E used in the present invention could inhibit apoptosis of the cultured cerebral cortical neurons and retinal ganglion cells induced by glutamic acid in an in vitro system. In other words, this mechanism is likely based on the inhibition of the activity of a glutamic acid receptor due to the formation of a complex of the lipoprotein with LRP1 (low density lipoprotein receptor-related protein 1) that is one of a lipoprotein receptor family.

The inventors of the present invention have reviewed, accordingly, using GLAST knockout mice, a model animal of glaucoma that is one of neurodegenerative diseases, as to whether the lipoprotein containing apolipoprotein E in the present invention could exhibit the neuroprotective effects in an in vivo system, as well. As a result, it was made clear that, despite lipoproteins contained in a large amount in the mouse vitreous humor, apoptosis of the retinal ganglion cells could be inhibited by the lipoprotein containing apolipoprotein E administered into the vitreous body. Surprisingly, the inventors of the present invention have confirmed the particular effects that the lipoprotein containing apolipoprotein E in the present invention could inhibit apoptosis of the retinal ganglion cells by a small amount of the lipoprotein containing apolipoprotein E in the present invention, administered into the vitreous body, despite the fact that a large amount of the lipoproteins with the apolipoproteins and original lipids connected thereto are present in the mouse vitreous body.

Therefore, the lipoprotein containing apolipoprotein E in the present invention is made clear that it can inhibit the neurodegeneration, particularly apoptosis of the nerve cells, among neurological diseases, not only in an in vitro system but also in an in vivo system.

The lipoprotein containing apolipoprotein E in the present invention is an apolipoprotein connected to lipoprotein, in which apolipoprotein E, one of apolipoproteins, is chemically associated with a lipid. As such lipoprotein containing apolipoprotein E, there may be include, for example, but not limited to glia-cell-derived lipoprotein containing apolipoprotein E, high density lipoprotein containing lipoprotein containing apolipoprotein E separated from the blood, reconstituted artificial lipoprotein containing apolipoprotein E. The high density lipoprotein may include the one containing a high density lipoprotein at a portion thereof or additionally containing apolipoprotein A1, J, D or the like. Hence, the high density lipoprotein separated from the blood is a fraction (liquid) of a variety of high density lipoproteins containing the lipoprotein containing apolipoprotein E.

Further, in the present invention, as described above, molecules related to the neuroprotective effects of the lipoprotein containing apolipoprotein E include the neuroprotective molecule exhibiting the neuroprotective effects upon activation of the lipoprotein containing apolipoprotein E and the neurodegeneration-inducing molecule exhibiting the neuroprotective effects upon inactivation (inhibition). As the neuroprotective molecules may include, for example, but not limited to lipoprotein receptors, phospholipase C, protein kinase Cδ. On the other hand, the neurodegeneration-inducing molecules may include, for example, but not limited to NMDA receptor, calcium and GSK3β. Among those, the lipoprotein receptors are receptors of a lipoprotein receptor family connected to the apolipoprotein E in the central nervous system and may include, for example, but not limited to—LRP1 receptor, LDL receptor, ApoER2 receptor, VLDL receptor, LR11 receptor, LRP4 receptor, LRP1B receptor, Megalin receptor, LRP5 receptor, and LRP6 receptor. Among these, LRP1 receptor is particularly preferred.

As described above, as the lipoprotein containing apolipoprotein E in the present invention can also inhibit the neurodegeneration of the neurological diseases particularly in an in vivo system, the lipoprotein containing apolipoprotein E according to the present invention may be applied as the prophylactic and therapeutic agent for neurological diseases, including neurodegenerative inhibitors such as nerve cell apoptosis inhibitor or the like, which use the lipoprotein containing apolipoprotein E as an active ingredient or the neuroprotective system utilizing the activation of the neuroprotective molecule and the inactivation of the neurodegeneration-inducing molecule, through a mediation of the lipoprotein receptor, or an action mechanism thereof. Moreover, such neurodegenerative inhibitors may include, for example, but not limited prophylactic or therapeutic agents for glaucoma and diabetic retinopathy.

The prophylactic and therapeutic agents for neurological diseases according to the present invention may be administered orally or parenterally. The dosage forms may include, for example, but not limited to tablets, capsules, fine granules, pills, troches, infusions, injections, eye drops, suppositories, ointments, and patches. When the prophylactic and therapeutic agent for neurological diseases according to the present invention is applied as a prophylactic and therapeutic agent for glaucoma or diabetic retinopathy, it is preferred to be administered through a parenteral route, particularly by injection into the vitreous body of the eye, and as the dosage form, an ophthalmological composition such as eye drops, ophthalmological injections or the like is preferred.

Upon administration of the prophylactic and therapeutic agents according to the present invention in vivo as infusion fluids, physiological saline may be formulated as needed with other water-soluble additives or liquid medicines. Such additives to be added to water may include, for example, but not limited to an alkali metal ion such as potassium and magnesium or the like, nutrients such as lactic acid, various amino acids, fat, carbohydrates such as glucose, fructose, saccharose, vitamins such as vitamin A, B, C, D, phosphate ions, chlorine ion, hormone agents, plasma proteins such as albumin, polymeric polysaccharides such as dextrin and hydroxyethyl starch. The concentration of the compound in such an aqueous solution may be preferably in the range of 10⁻⁷M to 10⁻⁵M.

The prophylactic and therapeutic agents according to the present invention may also be administered in vivo in the form of solid preparations which may include, for example, but not limited to—powders, fine granules, granules, microcapsules and tablets. Among the solid preparations, the tablets in a readily swallowable form are preferred.

As fillers and binders for forming the tablets, there may be used known ones including, for example, oligosaccharides and so forth. It is preferred that the diameter of the tablets is in the range of 2 to 10 mm and the thickness is in the range of 1 to 5 mm. They may be used in an admixture with other therapeutic agents.

To the solid preparations, various additives to be conventionally used may be formulated. The additives may include, for example, but not limited to stabilizers, surfactants, solubilizers, plasticizers, sweeteners, antioxidants, flavors, colorants, preservatives and inorganic fillers.

As the surfactants, there may include, for example, but not limited to anionic surfactants such as higher fatty acid soap, alkyl sulfate ester salts, polyoxyethylene alkyl ether sulfate salts, acyl N-methyltaurin salts, alkyl ether phosphate ester salts, N-acylamino acid salts or the like; cationic surfactants such as alkyltrimethylammonium chlorides, dialkyldimethylammonium chlorides, benzalkonium chloride or the like; amphoteric surfactants such as alkyldimethylaminoacetic acid betaines, alkylamido dimethylaminoacetic acid betaines, 2-alkyl-N-carboxy-N-hydroxyimidazolium betaines or the like; or nonionic surfactants such as polyoxyethylene types, polyvalent alcohol ester types, ethyleneoxide-propyleneoxide block copolymer.

The inorganic fillers to be formulated to improve swallowing or the like may include, for example, but not limited talc, mica and titanium dioxide.

The stabilizers may include, for example, but not limited to adipic acid and ascorbic acid. The solubilizers may include, for example, but not limited to surfactants such as sucrose esters of fatty acid, and stearylalcohols, asparagine, and arginine. The sweeteners may include, for example, but not limited to aspartame, hydrangea tea, licorice, and fennel.

The suspending agents may include, for example, but not limited to carboxyvinyl polymers. The antioxidants may include, for example, but not limited to ascorbic acid. The flavors may include, for example, but not limited to sugar flavors. The pH adjusting agents may include, for example, but not limited to sodium citrate.

The prophylactic and therapeutic agents according to the present invention may be administered in vivo usually in the dose of 1 mg to 40 mg and preferably 10 mg to 20 mg, per dosage and up to three times per day.

In a preferred embodiment of the present invention in which the prophylactic and therapeutic agents are applied in the form of eye drops or ophthalmological injections, the medicines may be combined with various ingredients including, for example, but not limited to pharmacologically active ingredients and pharmacologically active ingredients as long as they do not impede the effects of the present invention. As such ingredients, there may include, for example, but not limited to hyperemia-removing ingredients, ingredients of α-adrenergic stimulating agents, ingredients of anti-inflammatory agents, vitamins, amino acids, saccharides, steroid ingredients, ingredients of antihistaminic agents, and ingredients of antiallergic agents.

The hyperemia-removing ingredients may include, for example, but not limited to epinephrine and ephedrine. The ingredients of α-adrenergic stimulating agents may include, for example, but not limited to imidazoline derivatives (e.g. naphazoline and tetrahydrozoline), β-phenylethylamine derivatives (e.g. phenylephrine, epinephrine, ephedrine and methylephedine); the ingredients of anti-inflammatory agents may include, for example, but not limited to indomethacin, berberine, celecoxib and rofecoxib. active ingredients of antihistaminic agents and/or antiallergic agents may include, for example, but not limited to—chlorpheniramine, diphenhydramine and iproheptine; the vitamins may include, for example, but not limited to glutathione, vitamin C, E and B₆; the amino acids may include, for example, but not limited to leucine, isoleucine, valine, methionine, threonine, alanine, phenylalanine, tryptophan, lysine, glycine, asparagine and aspartic acid; the saccharides may include, for example, but not limited to a monosaccharide such as glucose, a disaccharide such as treharose, lactose and fructose, an oligosaccharide such as lactulose, raffinose, and pullulan; and a polysaccharide such as gum arabic, karaya gum, xanthan gum, guar gum and gum tragacanth; and a steroid may include, for example, but not limited to such as hydrocortisone and prednisolone.

The prophylactic and therapeutic agents for neurological diseases according to the present invention may be formulated to preparations using a formulation technology conventionally used in the technical field of the present invention. More specifically, the prophylactic and therapeutic agents for neurological diseases according to the present invention may be formulated into predetermined dosage forms by mixing a lipoprotein containing apolipoprotein E as the active ingredients of the present invention with excipients such as the stabilizers, plasticizers, antioxidants, sweeteners, flavors, preservatives, and inorganic fillers.

Moreover, the present invention can provide a prophylactic and therapeutic method for neurological diseases, which is able to prevent and treat the neurological diseases by applying to the neurological diseases, particularly neurodegenerative diseases, the prophylactic and therapeutic agents containing a lipoprotein containing apolipoprotein E according to the present invention as the active substance or the neuroprotective system utilizing the activation of the neuroprotective molecules and the inactivation of the neurodegeneration-inducing molecules, mediated with the lipoprotein receptor, as the action mechanism. More specifically, the prophylactic and therapeutic method according to the present invention includes an oral administration using dosage forms including, for example, but not limited to tablets, capsules, fine granules and pills; and a parenteral administration using of dosage forms including, for example, but not limited to infusions and injections.

A more detail of the present invention will be illustrated in the examples, but it is to be understood that the present invention is not whatsoever limited to the examples as described below. The following examples are described in an illustrative manner with the sole purpose to make the present invention more understandable and without any intention to limit the present invention in any respect.

### Example 1

### Materials

Among the materials to be used for the present invention, rabbit anti-LRP1 polyclonal antibody (R2629) was provided through the courtesy of Dr. D. K. Strickland of Medical School of Maryland University, Baltimore, Md.; Recombinant human apolipoprotein E3 and apolipoprotein E4 were purchased from Wako Junyaku Co., Ltd., Osaka. GLAST−/− mouse colony was established by Kumamoto University using mice obtained from Tokyo Medical and Dental University, Tokyo. All experimental procedures were carried out under consent by Animal Operations Committee of Kumamoto University.

(Primary Culture of Rat Retinal Ganglion Cells)

The primary culture of the retinal glia cells was carried out using two-day-old Sprague Dawley (SD) baby rats in accordance with a somewhat modified method (Hayashi et al., J. Biol. Chem. 2009) of the method of Barres et al. (Barres, et al., 1988). The isolated retinal glia cells (RGC) were suspended in a base culture medium of a RGC medium (1 mM glutamine, 5 μg/mL insulin, 60 μg/mL N-acetylcysteine, 62 ng/mL progesterone, 16 μg/mL putrescine, 40 ng/mL sodium selenite, 0.1 mg/mL bovine serum albumin, 40 ng/mL triiodothyronine, 0.1 mg/mL transferrin, 1 mM sodium pyruvate, 2% B27 supplement (Invitrogen, Carlsbad, Calif.), 10 μM forskolin (Sigma, St. Louis, Mo.), 50 ng/mL brain-derived neurotrophic factor (BDNF; PeproTech, Rocky Hill, N.J.), 50 ng/mL ciliary neurotrophic factor (CNTF; Peprotech), and 50 ng/mL basic fibroblast growth factor (bFGF; PeproTech). A 96-well plate was coated with poly-d-lysine (Sigma) and laminin (Sigma), and RGC was placed at the rate of 5,000 cells per well for the 96-well place, 5,000 cells per each culture insert for microdish, or 15,000 cells per dish for compartmentalized culture, followed by incubating for at least 10 days before experimentation.

### Example 2

### Production of Glia Cell-Derived Lipoprotein

The glia cell-derived lipoproteins were isolated from the medium adjusted for the glia cells. The glia cells were prepared from the cerebral cortex of 2-day-old SD-type rats and incubated in a Dulbecco's modified Eagle medium containing 10% fetal bovine serum. The glia cell medium was concentrated until the astrocytes accounted for more than 80% of the whole cells (Hayashi et al., J. Biol. Chem. 2009). The glia cells were then incubated in the RGC culture medium containing forskolin, BDNF, CNTF and bFGF. This culture medium was used as a medium for the production of glia cells.

### Example 3

This example is an experiment to investigate whether the glia cell-derived lipoprotein containing apolipoprotein E obtained in Example 2 inhibits apoptosis of the retinal glia cells by glutamic acid. The experiment was carried out in accordance with the procedures as will be described below as to whether the glia cell-derived lipoprotein containing apolipoprotein E obtained in Example 2 by incubating the retinal glia cells of the two-day-old baby rats obtained by the primary culture for 14 days or more in Example 1 possessed the action of inhibiting apoptosis of the retinal glia cells by glutamic acid. The results are shown in FIG. 1. In the figure, Cant represents a non-treated group, Glu represents a group with 300 μM glutamic acid added, and +glial LP represents a group with 300 μM glutamate+glia-cell-derived lipoprotein containing apolipoprotein E added. Glial LP was added in the concentration of 1 μg cholesterol/mL. After treatment, the rate of apoptosis-like cells was computed by observing the aggregation of the nuclei (a: nuclear staining with Hoechst 33342) or the sustained impermeability of esterase activity/membrane (b: calcein AM/Propidium Iodide staining) in 24 hours. As a result, it was found that a lipoprotein containing the glia cell-derived apolipoprotein E significantly inhibited an apoptosis of the retinal ganglion cells induced by glutamic acid (**p<0.05: Glu vs. Glu+glial LP.)

### Example 4

This example is an experiment to investigate whether the lipoprotein containing the glia-cell-derived apolipoprotein E obtained in Example 2 can inhibit an apoptosis of the cerebral cortical neurons by glutamic acid. The experiment was carried out in accordance with substantially the same procedures as Example 3 as to whether the lipoprotein containing the glia-cell-derived apolipoprotein E obtained in Example 2 by incubating the cerebral cortical neurons subjected to the primary culture of sixteen-day-vivipary rats for 6 days had the action of inhibiting apoptosis of the retinal glia cells by glutamic acid. The results are shown in FIG. 2. In the figure, Cont represents a non-treated group, Glu represents a group with 300 μM glutamic acid added, and +glial LP represents a group with 300 μM glutamic acid+lipoprotein containing the glia-cell-derived apolipoprotein E. Glial LP was added in the concentration of 1 μg cholesterol/mL. After treatment, the rate of apoptosis-like cells was computed by observing the aggregation of the nuclei by the nucleus staining with Hoechst 33342 in 24 hours. As a result, the glia cell-derived lipoprotein containing apolipoprotein E was found to significantly inhibit apoptosis of the cerebral cortical neurons to be induced by glutamic acid (**p<0.0001: Glu vs. Glu+glial LP)

### Example 5

### Production of Plasma HDL

Mouse plasma HDL or rat plasma HDL was isolated from the ventral aortic blood of C57BL/6J mice or SD-type rats, respectively.

### Example 6

This example was carried out in substantially the same manner as Example 3 to investigate whether the high density lipoprotein (HDL) isolated from the rat blood in Example 5 inhibited apoptosis of the retinal glia cells by glutamic acid. As a result, the HDL isolated from the rat blood inhibited the apoptosis induced in the method similar to Example 3 (See FIG. 3; *p<0.05: Glu vs. Glu+HDL). The HDL was added in the concentration of 1 μg cholesterol/mL. The apoptosis-like cells were computed by the nucleus staining with Hoechst 33342.

### Example 7

### Production of Reconstituted Artificial Apolipoprotein E-Containing Lipoprotein

The reconstituted lipoprotein containing apolipoprotein E (E-LP) was produced in accordance with the procedures as described above (Hayashi et al., J. Neurosci. 2007). The reconstituted lipoprotein containing apolipoprotein E was composed of 1-palmitoyl-2-oleyl-glycerophosphocoline (POPC; P3017, Sigma), cholesterol (C3045, Sigma) and recombinant human apolipoprotein E in the molar ratio of 100:10:1 or 100:0:1. A solution containing the preparatory medium, plasma or the reconstituted lipoprotein containing apolipoprotein E was subjected to a discontinuous sucrose gradient. The composition of the used solution was 3 mL at the density of 1.30 g/mL, 3 mL at the density of 1.2 g/mL, 3 mL at the density of 1.1 g/mL, and 6 mL at the density of 1.006 g/mL. The sucrose gradient was centrifuged at 100,000 g for 72 hours at 4° C. using SRP28SA1 rotor (Hitachi, Tokyo, Japan). Ten fractions (1.5 mL) were collected from the top of the gradient and subjected to immunoblotting for apolipoprotein E in the manner as will be described below. The fractions containing apolipoprotein E (typically fractions 5-7) were combined and filtered with Amicon Ultra filter (molecular weight of 100 kDa or 50 kDa cut off; Millipore, Bedford, Mass.). The reconstituted lipoprotein was adjusted to the cholesterol concentration (2 μg/mL) for the HDL and to the protein concentration (100 ng/mL) for the reconstituted lipoprotein. The cholesterol and protein concentrations were measured by LabAssay cholesterol kit (Wako) and BCA protein assay kit (Thermo Fisher Scientific Inc., Rockford, Ill.), respectively.

### Example 8

This example was carried out in the same manner as in Example 3 as to whether the reconstituted lipoprotein containing human apolipoprotein E3 (LP) obtained in Example 7 could inhibit apoptosis of the retinal glia cells by glutamic acid. As a result, the reconstituted lipoprotein containing the human apolipoprotein E3 (LP) inhibited the apoptosis induced by the method similar to Example 3 (See FIG. 4; *p<0.05: Glu vs. Glu+LP). The human apolipoprotein E3 was added in the concentration of 100 ng protein/mL. The apoptosis-like cells were computed by the nucleus staining with Hoechst 33342.

### Example 9

This example was carried out in the procedures similar to those of Example 3 as to whether the neurodegeneration-inducing molecule such as NMDA receptor, calcium, calpain, calcineurin and caspase was involved in the apoptosis of the retinal glia cells induced by glutamic acid. The results are shown in FIG. 5 (*p<0.05: Glu vs. Glu+MK801, ALLN, FK506 or Z-VAD). In the figure, (a) indicates the inhibition of apoptosis of N-methyl-D-aspartic acid receptor (NMDA receptor) by an inhibitor MK 801 (10 μM). Figure (b) indicates no apoptosis induction by glutamic acid in a culture liquid containing no calcium. Figure (c) indicates the inhibition of apoptosis by calpain inhibitor ALLN (1 μM), but no inhibitory effect by dimethylsulfoxide (DM) with ALLN dissolved therein. Figure (d) indicates the inhibition of apoptosis by calcineurin inhibitor FK 506 (1 μM). Figure (e) indicates the apoptosis inhibition by caspase inhibitor Z-VAD-FMK (20 μM). The apoptosis-like cells were computed by the nucleus staining with Hoechst 33342.

### Example 10

This example indicates that the reconstituted lipoprotein containing human apolipoprotein E3 (LP) protected the retinal glia cells by a mediation of LRP1 (low density lipoprotein receptor-related protein 1) and GSK3β was involved in its mechanism (see FIG. 6; *p<0.05: Glu+LP vs. Glu+LP+anti-LRP1). FIG. 6a shows that the neuroprotective effects of the reconstituted lipoprotein containing the human apolipoprotein E3 were inhibited by a LRP1 antibody (10 μg/mL) and not by normal IgG. The apoptosis-like cells were computed by the nucleus staining with Hoechst 33342. FIG. 6b shows the recovery of the phosphorylation level of glycogen synthase kinase 3β (GSK3β) by the reconstituted lipoprotein containing human apolipoprotein E3 (LP). The retinal glia cells were collected in 18 hours after treatment with glutamic acid, followed by Western blotting in which a rabbit anti-phosphorylated GSK3β (p-GSK3β) antibody and a rabbit anti-GSK3β antibody were used.

### Example 11

This example indicates that the reconstituted lipoprotein containing apolipoprotein E3 (LP) promotes the formation of a complex of LRP1 and NMDA receptor (see FIG. 7). LP was added to a culture liquid of the retinal glia cells and the cells were collected in 15 minutes, followed by the immune precipitation as will be described below. For the immune precipitation, a rabbit anti-NMDA receptor 2B antibody was used, and the Western blotting was performed for LRP1 and NMDA receptor 2B.

### Example 12

This example is an experiment to investigate whether the reconstituted lipoprotein containing apolipoprotein E3 (LP) inhibits apoptosis of the retinal glia cells of a GLAST knockout mouse as a normal-pressure glaucoma model animal. This experiment was carried out by administering LP (100 ng protein/mL) or a phosphate buffer (a control group) to the vitreous body of a three-week-old GLAST knockout mouse (3w KO). The eyeball was removed at the time of six weeks, followed by fixing with paraffin, staining with hematoxylin-eosin, and counting the retinal glia cells on the retina.

This result revealed that the mouse LP inhibited apoptosis of the retinal glia cells despite the mouse LP was present in a large amount in the vitreous body. This mechanism is considered to be caused by the formation of a complex of the lipoprotein conjugated to LRP1, one member of the lipoprotein receptor family and the inhibition of the activity of a glutamate receptor.

(Injection of Apoprotein E-LP into Vitreous Body and Collection of Vitreous Humor)

Three-week-old GLAST+/− or GLAST−/− mice were anesthetized by intraperitoneal injection of 50 mg/kg of pentobarbital sodium. For the injection into the vitreous body, the vitreous body of one of the eyes was injected with 1 μl of apoprotein E-LP (1.5 μg protein/mL) or HDL (30 μg cholesterol/mL) by a 33-gauge Nanopass® needle (Terumo, Tokyo, Japan) mounted on a polyethylene tube (SPS, Natume, Tokyo, Japan) and a 10 μL Hamilton syringe (Hamilton, Bonaduz, Switzerland) while the vitreous body of the other eye was injected with the same amount of PBS. This operation was performed with care under a stereoscopic microscope in order to cause no damages of the crystalline lens and the retina. The vitreous humor was collected using the same needle as used for injection into the vitreous body.

(Histological Studies of Mouse Retina)

The eyeball of a six-week-old mouse was removed and fixed overnight at 4° C. using Super Fix (KY-500, Kurabo. Osaka), followed by removing the cornea and the crystalline lens under a stereoscopic microscope. The retina with the sclera was embedded in paraffin. Thereafter, 4 μm paraffin sections of the retina were prepared and then stained with hematoxylin and eosin. Five sections were selected from each of the continuous sections and the number of the cells within the glia cell layer was counted in a range extending from one end of the retinal section to the other end through the optic nerve. More than 2,000 cells were counted on 10 sections of each retina.

### Example 13

### Immune Blotting

The immune blotting used in the above example was carried out in a manner as will be described below. Specifically, the lipoprotein obtained in each of the above examples was dissolved in 62.5 mM Tris-HCl (pH 6.8), 10% glycerol, 2% sodium dodecyl sulfate (SDS) and 5% β-mercaptoethanol (a sample buffer), and the resulting mixture was boiled for 5 minutes. The resulting protein was separated by electrophoresis using 0.1% SDS-containing polyacrylamide gel and then transferred to a polyvinylidene difluoride membrane. Thereafter, the membrane was incubated at room temperature for 1 hour together with skim milk with TBS-T (10 mM Tris-HCl (pH 7.4), 150 mM NaCl and 0.1% Tween 20 added thereto, followed by probing overnight using a primary antibody in TBS-T with 5% fetal bovine albumin added thereto. Then, the membrane was further probed at room temperature for 1 hour using peroxidase-conjugated goat anti-rabbit IgG (Thermo), goat anti-mouse IgG (Thermo) or mouse anti-goat IgG (Thermo). The immunoreactive protein was visualized by chemiluminescence (GE Healthcare, Buckinghamshire, UK) or Super Signal West Dure (Theremo). The primary antibodies used are as follows: mouse anti-β-actin (a5441, dilution 1:10,000, Sigma), goat anti-human apoprotein E (k74190g, dilution 1:5000, Biodesign, Saco, Me.), goat anti-mouse apoprotein E (sc-6384, dilution 1:1000, Santa Cruz), rabbit anti-human GSK3b and phospho-Ser 9-GSK3β (9315 and 93365, dilution 1:1000, Cell signaling Technology, Danvers, Mass.), goat anti-human Bm-3a (sc-31984, dilution 1:1000, Santa Cruz), rabbit anti-human LRP1 (2703-1, dilution 1:1000, Epitomics), mouse anti-human LRP1 (545503, dilution 1:1000, R&D systems, Minneapolis, Minn.), rabbit anti-bovine phospholipase Cγ1 (sc-81, dilution 1:1000, Santa Cruz), and mouse anti-rat NMDAR2B (610416, dilution 1:1000, BD Biosciences, San Jose, Calif.).

### Example 14

### Immunocytochemistry of RGC

The cultured RGC was washed twice with phosphate buffer-physiological saline (PBS) and fixed with acetone for 10 minutes at 4° C. The RGC was then blocked at room temperature for 1 hour using a PBS solution of 1% bovine serum albumin and 5% goat serum, followed by incubation at room temperature for 1 hour using a PBS solution (containing 1% bovine serum albumin and 5% goat serum) of rabbit anti-human LRP1 (dilution 1:2000, Epitomics), mouse anti-NMDAR2B (32-0700, dilution 1:500, Invitrogen), and mouse anti-rat cytochrome c (556432, dilution 1:3000, BD Biosciences). The resulting cells were then washed three times with PBS and incubated at room temperature for 1 hour using Alexa Fluor 488-conjugated goat anti-rabbit IgG (dilution 1:200, Invitrogen), Alexa Fluor 488-conjugated goat anti-mouse IgG (dilution 1:200, Invitrogen) or Alexa Fluor 594-conjugated anti-mouse IgG (dilution 1:200, Invitrogen). The resulting RGC was washed three times with PBS and allowed to start an immunoresponse using Fluormount/Plus (Japan Tanner, Osaka, Japan). For the staining of mitochondria, the RGC was incubated at 37° C. for 30 minutes using 2 nM MitoTracker Red CMXRos (Invitrogen) one day before the start of the experiment. The pictures were taken by Olympus IX71 Microscope (Tokyo, Japan) or Olympus FV500 cofocal microscope.

### Example 15

The induction and detection of apoptosis in RGC were performed in the manner as described below. Specifically, RGC was washed twice with Hanks' balanced salt solution (HBSS) containing 2.4 mM CaCl₂ and 20 mM HEPES, but not containing magnesium, and incubated at 37° C. for 2 hours in a medium prepared by adding 300 μM glutamate and 10 μM glycine to the above Hanks' balanced salt solution. After treatment with glutamate, RGC was incubated at 37° C. for 22 hours in a RGC culture medium containing no forskolin, BDNF, CNTF and bFGF. In order to detect apoptosis, RGC was stained with 1 μg/mL of Hoechst 33342 (346-07951, Dojindo, Kumamoto, Japan) or 1 μg/mL of calcein-AM/propidium iodide (341-07381, Dojindo). The fluorescence images were observed by Olympus IX71 microscope. The fragmented or aggregated nuclei stained with the above Hoechst reagent were counted as apoptotic nuclei, and round and smooth nuclei were counted as sound nuclei. As the sound nuclei were not stained with propidium iodide, the nuclei stained with propidium iodide were counted as apoptotic nuclei. More than 300 nuclei were counted in each group.

### Example 16

The inflow of calcium into RGC was confirmed in the manner as described below. Specifically, RGC was incubated on a microdish at least for 14 days, followed by incubation at 37° C. for 30 minutes using 3 μM Fluo-8 acetoxymethyl ester (AAT Bioquest, Sunnyvale, Calif.). The resulting cells were washed twice with 500 μl of the above HBSS, and 300 μM glutamate and 10 μM glycine were poured. The fluorescence images were taken at every 500 msec by ORCA-R2 digital camera (Hamamatsu Photonics, Hamamatsu, Japan) and analyzed by MetaFluor fluorescence ratio imaging software (Molecular Devices, Sunnyvale, Calif.).

### Example 17

The compartmentalized culture of RGC was carried out in the manner as described below. For the compartmentalized culture, RGC was prepared in accordance with the method as described in the known literature document (Hayashi et al., J. Biol. Chem. 2004). After a microdish was coated with poly-d-lysine and laminin, the surface of the microdish was scratched to form 20 parallel grooves, and a TEFLON (registered trade mark) three-compartment divider was mounted on the dish with silicon grease. In the central compartment, a RGC culture medium containing 25 ng/mL BDNF and 25 ng/mL CNTF was poured, and the RGC cells were inoculated at a rate of 10,000-15,000 cells per dish. In each of the compartments on the both sides, a RGC culture medium containing 75 ng/mL BDNF, 25 ng/mL CNTF and 50 ng/mL bFGF was poured. The RGC axons were invaded into the compartments on the both sides within five days beyond beneath the silicon grease. Before the start of this experiment, the compartmentalized culture was performed at least for 14 days.

### Example 18

The immune precipitation was carried out in the manner as will be described below. Specifically, the co-immunoprecipitation was performed in accordance with procedures of May et al. (May et al., 2004) by using a mixed solution prepared by adding a RGC dissolved solution to a mixed solution of a complete EDTA-free protease inhibitor cocktail (Roche, Mannheim, Germany) and a PhosSTOP phosphatase inhibitor cocktail (Roche) and a mixture of 10 mM Tris-HCl (pH7.4), 150 mM NaCl, 1 mM MgCl₂, 1 mM CaCl₂ and 1% Triton X-100. RGC was washed once with HBSS and collected on a 96-well plate using 40 μl per well of a dissolving buffer. The dissolved solutions from wells 9-12 were combined in each group. The RGC dissolved solution was passed fifteen times through a 22-gauge needle and then centrifuged at 15,000×g for 15 minutes at 4° C. The resulting supernatant was pretreated at 4° C. for 1 hour with 40 μl of 50% equilibrated protein G-Sepharose (GE Healthcare, Buckinghamshire, UK), and the Sepharose beads were removed by centrifugation. After an addition of rabbit anti-LRP1 antibody (dilution 1:200; 2703-1, Epitomics, Burlingame, Calif.) or rabbit anti-NR2B antibody (dilution 1:200; AB1557, Millipore), the RGC lysate was rotated at 4° C. for 12 hours. To the resulting lysate, 40 μl of 50% equilibrated protein G-Sepharose was added, followed by rotation at 4° C. for another 1 hour. The Sepharose beads were washed three times with a lysate buffer containing 0.1% Triton X-100. After 30μ of the sample buffer was added, the Sepharose beads were boiled for 5 minutes to perform immune blotting. The resulting supernatant was then used for SDS-polyacrylamide gel electrophoresis and immune blotting.

### Example 19

This example was carried out to investigate an apoptosis induction of RGC with glutamic acid and glycine.

FIG. 9A shows the fragmented or shrunken nuclei in RGC, which were detected by the Hoechst staining after a control (C; HBSS) or in 24 hours after treatment with glutamic acid only (Glu(-); 300 μM glutamate), glycine only (Gly(-); 10 μM glycine) or glutamic acid+glycine (Glu; 300 μM glutamate+10 μM glycine). Data are expressed as average+/−SE values obtained by independent four experiments (*: p<0.001 (C vs Glu)). FIG. 9B shows the fragmented or shrunken nuclei in RGC, which were detected by the Hoechst staining in 24 hours after treatment with a control (C; HBSS) or with glutamic acid+glycine (Glu; 300 μM glutamate+10 μM glycine) which in turn was not washed with HBSS or washed with HBSS once, three times or three times, each for 15 minutes. FIG. 9C shows fluorescent images of RGC stained with Annexin V-EGFP, propidium iodide, and Hoechst in 12 hours after treatment with a control or glutamic acid+glycine (Glu; 30 μM glutamate+10 μM glycine). FIG. 9D shows the ROC immunostained with anti-cytochrome c (Cyto C) in 12 hours after treatment of the RGC with 2 nM MitoTracker Red (Mito) and then with a control or Glu. The scale bar is 20 μm.

### Example 20

This example was carried out to examine a substance involved in a glutamate-induced cytotoxicity in RGC.

The fragmented or shrunken nuclei were detected in RGC by the Hoechst staining after a control (HBSS) or in 24 hours after treatment with glutamate+glycine (Glu; 300 μM glutamate+10 μM glycine).

FIG. 10A is a figure showing the substances involved in the glutamate-induced cytotoxicity, which was obtained by incubating the RGC cells with glutamate in the absence of Ca2+(no Ca2+) or in the presence of Ca2+(Ca2+). FIG. 10B shows the case in which the RGC was treated with 10 μM MK801 (NMDA receptor inhibitor); FIG. 10C shows the case in which treated with 1 μM ALLN (calpain inhibitor); FIG. 10D shows the case in which treated with 1 μM FK506 (calcineurin inhibitor); FIG. 10E shows the case in which treated with 200 μM MK801 (Bax-inhibitory peptide V5; BIP-V5; Baxinhibitor); and FIG. 10F shows the case in which treated with 20 μM Z-VAD-fink (Z-VAD; caspase inhibitor). In the case of treatment with Glu, the RGC was treated with dimethyl sulfoxide. In FIGS. 10B-F, *: p<0.001 (Glu vs. Glu+inhibitor). Data were expressed as average+/−SE values obtained by independent four to six experiments.

### Example 21

The present inventors have previously reported that the apoptosis induction of RGC was inhibited with the glia-derived E-LP by removal of nutritive additives. This example was carried out, accordingly, to investigate whether a lipoprotein can prevent the glutamate-induced apoptosis.

The glia-derived E-LP, plasma HDL and reconstituted E-LP were extracted in the following way. First, glia was extracted from the cerebral cortex of a two-day-old Sprague Dawley rat, digested with 0.25% trypsin, and incubated in a Dulbecco's modified Eagle medium containing 10% fetal bovine serum. The glia was then incubated for 3 days in the same medium as used for the RGC (without containing forskolin, brain-derived neurotrophic factor, ciliary neurotrophic factor and basic fibroblast growth factor). The resulting culture fluid was centrifuged at 1,000×g for 10 minutes, and the resulting supernatant was used as a glia adjustment medium. The mouse or rat HDL was isolated from the blood collected from the ventral aorta of C57BL/6.1 mouse or Sprague Dawley rat. The reconstituted E-LP was prepared by procedures as described in literature documents. To the reconstituted E-LP, there were added 1-palmitoyl-1-oleoyl-glycerophosphocoline, cholesterol and recombinant human apoE at the respective rate of 100/10/1 or 100/0/1. 1-Palmitoyl-1-oleoyl-glycerophosphocoline (2.71 mg) was added singly or in combination with cholesterol (0.14 mg), followed by dissolving in chloroform and evaporating under nitrogen gas. Thereafter, 400 μl of 10 mM Tris-HCl (pH 7/4) containing 0.9% NaCl was added, the resulting mixture was incubated for 1 hour on ice, and 15 mg/mL of sodium cholate (100 μl) was added. After the resulting mixture was incubated for 2 hours on ice, it was admixed with recombinant apoE3 or E4 (1 mg), followed by incubation for 1 hour on ice. Thereafter, Bio-Beads (100 mg; Bio-Rad, CA) were added, rotated at 4° C. for 3 hours, and then removed. This mixture contained the reconstituted lipoprotein. The glia adjustment medium, the plasma or the reconstituted lipoprotein was centrifuged at 100,000×g for 72 hours at 4° C. by intermittent sucrose gradient with the solution below: density 1.30 g/mL (3 mL), density 1.2 g/mL (3 mL), density 1.1 g/mL (3 mL) and density 1.006 g/mL (6 mL). Ten fractions (1.5 mL) at the gradient top were collected and subjected to immunoblotting for apoE. After the apoE-containing fractions were concentrated, they were adjusted to the cholesterol concentration (2 μg/mL) for the glia-derived E-LP and HDL as well as to the protein concentration (100 ng/mL) for the reconstituted lipoprotein. The cholesterol concentration and the protein concentration of the lipoprotein were measured using LabAssay cholesterol kit (Wako Jyunyaku) and BCA protein assay kit (Termo Fisher Scientific, IL). α2-macroglobulin was activated by treatment with 100 mM methylamine at room temperature for 1 hour.

The apoptosis of RGC was measured in the following manner. The RGC subjected to the primary incubated was incubated two times at 37° C. for 15 minutes using Hanks' Balanced Salt Solution (HBSS) containing 2.4 mM CaCl2 and 20 mM 4-(2-hydroxyethyl)-pyperazine-1-ethanesulfonic acid (HEPES) but containing no magnesium and then washed. As the NMDA receptor was blocked, the magnesium was removed from the washing solution. Thereafter, the RGC was treated or not treated with 300 mM glutamate+10 mM glycine (a co-activating agent for the NMDA receptor) in HBSS containing 2.4 mM CaCl2 and 20 mM HEPES (without magnesium) and then incubated at 37° C. for 2 hours. A control (HBSS containing 2.4 mM CaCl2 and 20 mM HEPES (without containing magnesium)) or the RGC treated or not treated with glutamic acid was incubated at 37° C. for 22 hours in the same medium as used for the RGC (without forskolin, brain-derived neurotrophic factor, ciliary neurotrophic factor and basic fibroblast growth factor). In order to detect apoptosis using Hoechst 33342 (Dojin Kagaku), the RGC was incubated for 15 minutes together with 1 μg/mL of Hoechst 33342. Fluorescent images (6 images/well) were photographed randomly by IX71 fluorescence microscope. For each treatment, photographs of at least 12 images were taken per well of 96 wells. The fragmented or shrunken nuclei stained by the Hoechst staining were counted as apoptotic nerve cells, and round or smooth nuclei were counted as normal nerve cells. For each treatment, more than 300 nerve cells were counted blindly. The detection of apoptosis with Annexin V-EGFP Apoptosis Detection Kit (MBL) containing Annexin V-EGFP, propidium iodide iodid and a connecting buffer was performed in accordance with the manufacturer's manual. During the initial stage of apoptosis, phosphatidyl serine was being exposed on the outside leaflet of a plasma membrane and then stained with Annexin V. Propidium iodide stained the necrotic cells and the apoptotic cells at the terminal stage. On the other hand, normal cells were not stained with either of the reagents.

In other words, the experiment was carried out to investigate whether these lipoproteins protected the RGC from the glutamate-induced cytotoxicity using the glia-derived lipoprotein (2 μg cholesterol/mL) extracted from the glia adjustment medium (GLP) and HDL (2 μg cholesterol/mL) extracted from rat plasma. This experiment was performed to investigate whether fragmented or shrunken nuclei were detected in the RGC by the Hoechst staining after a control (HBSS) or in 24 hours after treatment with glutamic acid+glycine (Glu; 300 μM glutamate+10 μM glycine). As a result, the apoE-containing lipoproteins of these two types protected the RGC from the glutamic acid-induced cytotoxicity (FIG. 11A).

The recombinant apoE, cholesterol and the reconstituted E-LP protected the RGC in a dose-dependent manner (FIG. 11B).

It was further found that, as a result of investigations regarding the ingredients of E-LP needed for protection of the nerves, the association of apoE with a lipid was needed (FIG. 11C). Although neither cholesterol (11 ng/mL) alone nor a mixture of cholesterol with phosphatidyl choline (11 ng/mL) promoted the existence, however, apoE (100 ng protein/mL) possessed the neuroprotective effects when connected to phosphatidyl choline (11 ng cholesterol/mL). It is preferable that the molar ratio of phosphatidyl-choline/cholesterol/apoE is 100±10/10±1/1±0.1 (90-110/9-11/0.9-1.1).

Moreover, the isoforms of human apoE3 and apoE4 exerts an influence on the neurodegeneration, particular neurodegeneration in Alzheimer's disease. According to the experiments conducted by the present inventors of the present invention, however, no difference was recognized between the human lipoprotein containing apoE3 and apoE4 regarding the neuroprotective effects in the RGC treated with glutamic acid.

FIG. 11A shows immunoblots for apoE in the lipoprotein extracted from the glia adjustment medium (GLP), apoE in the HDL extracted from the rat plasma, and the reconstituted human lipoprotein containing apoE (E-LP). After the RGC was incubated for 15 minutes with GLP, HDL, or E-LP, Glu was added. *: <0.005 (Glu vs. Glu+GLP, Glu+HDL or Glu+E-LP).

FIG. 11B shows the dose-dependent protective effects of E-LP on the Glu-induced neurotoxicity. After the RGC was incubated with E-LP for 15 minutes, Glu was added. * and **: p<0.005 and p<0.0001 (Glu vs. Glu+E-LP).

FIG. 11C shows the cases in which the RGC was incubated with lipid-free apoE (100 ng protein/mL), cholesterol (chop (11 ng/mL), phosphatidyl choline+cholesterol (PC+chol) liposome (11 ng cholesterol/mL), PC+apoE liposome (100 ng protein/mL), apoE-containing E-LP, cholesterol and phosphatidyl choline (100 ng protein/mL and 11 ng cholesterol/mL, respectively), or HDL from mouse plasma (MsHDL) (2 μg cholesterol/mL), followed by addition of Glu, respectively. * and **: p<0.001 and p<0.0001 (Glu vs. Glu+lipoprotein).

FIG. 11D shows the cases in which the RGC was incubated with the reconstituted apoE3-containing liposome (E3-LP) or apoE4-containing liposome (E4-LP) (100 ng protein/mL) for 15 minutes, followed by addition of Glu, respectively. *: p<0.001 (Glu vs. Glu+E3-LP or E4-LP).

### Example 22

This example was to examine the inhibition of an increase in the intracellular Ca2+ caused by an interaction between LRP1 and NMDA receptor by E-LP.

RGC was labeled with Fluo-8 acetoxymethyl ester for 30 minutes, and Glu (300 μM glutamate+10 μM glycine) was added thereto. Fluorescent ratio images were as represented by colors indicated by the color index shown at the lower side of FIG. 12A. The color was represented as Ratio 0 and Ratio 2 corresponding to the base fluorescent intensity (Ratio 1) before Glu stimulation. The panels on the left and right sides indicate Glu and Glu+E-LP, respectively. This data was obtained from one of the eight experiments which demonstrated similar results. The scale bar is 80 μm.

FIGS. 12B and C show variations of fFuo-8 fluorescence represented by ΔF/F0. In the figures, F0 indicates the base fluorescence intensity before Glu stimulation. * and **: p<0.005 and p<0.0001 (Glu vs. Glu+E-LP). RGC was incubated for 15 minutes together with 100 ng protein/mL of E-LP, 10 μM MK801, 100 ng protein/mL E-LP+10 μM MK801, 100 ng protein/mL E-LP+10 μg/mL anti-LRP1 antibody or 100 ng protein/mL E-LP+10 μg/mg IgG, followed by addition of Glu thereto in an amount indicated. The data is represented as average±SE values of the results of 6 to 8 experiments showing similar results. FIG. 12D shows a rate of the fragmented or shrunken nuclei detected by the Hoechst staining after a control (HBSS) or in 24 hours after treatment with glutamate+glycine (Glu; 300 μM glutamate+10 μM glycine). To the RGC, E-LP (100 ng protein/mL), anti-LRP1 antibody (10 μg/mL) or E-LP IgG (10 μg/mg) was added over 15 minutes and Glu was then added. *: p<0.01 (Glu+E-LP vs Glu+E-LP+anti-LRP1). This data is an average±SE value of 4 experiments. FIGS. 12E and F show results of immunoprecipitation using an antibody of LRP1 (E) and NMDA receptor subunit NR2B (F), respectively, from the RGC culture fluid untreated or treated with E-LP. The immunoprecipitated material (pellet) and the supernatant were probed with the LRP1, NR2B or NR2A antibodies. This data is an average±SE value of one of three experiments showing similar results.

### Example 23

This example was to examine the prophylactic effects of E-LP on the glutamate-induced neurotoxicity by phospholipase, protein kinase Cδ and GSK3β.

FIG. 13A shows rates of the fragmented or shrunken nuclei detected by the Hoechst staining after a control (HBSS) or in 24 hours after treatment with glutamate+glycine (Glu; 300 μM glutamate+10 μM glycine). RGC was incubated for 15 minutes together with E-LP (100 ng protein/mL) or E-LP+U (5 μM U73122, phospholipase C inhibitor), followed by addition of Glu thereto. This data is an average±SE value of five experiments. *: p<0.005 (Glu+E-LP vs Glu+E-LP+U).

FIG. 13B shows protein kinase Cδ knocked out with PKCδ siRNA in RGC. RGC was incubated for 6 days together with 300 nM negative control (NC) or PKGδ siRNA and the PKCδ was detected by immunoblotting. β-Actin was used as an internal control. The fragmented or shrunken nuclei were knocked out with the negative control (NC) or PKCδ siRNA, followed by treatment with C, Glu or Glu+E-LP and detection by the Hoechst staining after 24 hours. This data is an average±SE value of five experiments. *: p<0.05 (Glu+E-LP+NC vs Glu+E-LP+PKCδ).

FIG. 13C shows the RGC collected in 16 hours after treatment with C, Glu or Glu+E-LP. The RGC was immunoblotted with an antibody to GSK3β phosphorylated with Ser9 (p-GSK3β) or total GSK3β. The quantitation of the Ser9 phosphorylation of GSK3β was indicated from four experiments. *: p<0.001 (Glu vs Glu+E-LP).

### Example 24

This example was to examine the glutamate-induced apoptosis in a cell body compartment (not a tip axon compartment). On the upper panel of FIG. 14A, a phase contrast image of one track shows the localization of the cell body in the cell body compartment and the existence of the axon on the right axon compartment. The lower-end panel shows the RGC stained with anti-LRP1 antibody or anti-NR2B antibody. The scale bar is 50 μm.

FIGS. 14B and C show the fragmented or shrunken nuclei detected by the Hoechst staining in 24 hours after treatment of the cell body (B) or the axon (C) with glutamate (Glu; 300 μM glutamate+10 μM glycine). RGC was incubated for 15 minutes together with 100 ng protein/mL of E-LP, followed by addition to the cell body (B) and the axon (C), respectively. *: p<0.005 (Glu vs Glu+E-LP). This data is an average±SE value of four experiments.

### Example 25

This example was to examine the recovery of RGC existence by E-LP in Glast+/− and Glast−/− mice. FIGS. 15A and B show the results obtained by immunoblotting the retina of three-week-old (3W) or six-week-old (6W) Glast−/− mice (injected with 1 μl of PBS or 1 μl of 1.5 μg protein/mL E-LP) with anti-Bm-3a antibody or anti-β-actin antibody (A) or an antibody to GSK3β phosphorylated with Ser9 (p-GSK3β) or total GSK3β (B). The quantitation of β-actin vs. Brn-3a (A) and Ser9-phosphorylated GSK3β vs. total GSK3β (B) was conducted from five and six experiments, respectively. *: p<0.05 (PBS vs E-LP; six-week-old Glast−/− mouse).

FIG. 15C shows the results of hematoxylin-eosin staining of the retina segments from Glast+/− and Glast−/− mice (3-week-old or 6-week-old) injected with 1 μl of PBS, 1 μl of 1.5 μg protein/mL E-LP or 30 μg cholesterol/mL HDL. The arrowhead shows the glia cell layer (GCL) of the retina. The scale bar is 40 μm. The data is obtained from one retina segment representative of eight segments indicating the similar results.

FIG. 15D shows the RGC numbers quantitated in 6-week-old wild-type mouse (+1+), three-week-old or six-week-old Glast+/− mouse (+/−) or Glast−/− mouse (−/−), Glast+/− and Glast−/− mice, each non-injected or injected with 1 μl of PBS, 1 μl of 1.5 μg protein/mL E-LP or 30 μg cholesterol/mL HDL. The data were obtained from the results of eight experiments. *: p<0.05 (PBS vs E-LP or HDL; 6-week-old Glast−/− mouse). #: p<0.05 (PBS vs E-LP or HDL; 6-week-old Glast+/− mouse).

### Example 26

This example shows a comparison of the amount of apolipoprotein E in the vitreous body of Glast+/+ and Glast−/− mice with the amount of apolipoprotein E in E-LP injected in the vitreous body. FIG. 16 shows the results of immunoblotting of 5 μl of vitreous humor of Glast+/+ and Glast−/− mice and 5 μl of 100 ng/mL E-LP.

### Example 27

This example was to examine that E-LP inhibited the inhibitory effects of α2-macroglobin. FIGS. 17A and B show the results obtained by immunoblotting the retina or vitreous humor from three-week-old or six-week-old Glast+/+ and Glast−/− mice with an antibody to apoE, LRP1, β-actin, α2-macroglobin (a2M), and albumin. The arrowhead indicates a2M. FIG. 17A shows the results of four experiments conducted for the quantitation of apoE with respect to β-actin. *: p<0.05 (3-week-old Glast+/+ mouse vs retina of 3-week-old Glast−/− mouse). FIG. 17B shows the results of three experiments conducted for the quantitation of apoE and a2M with respect to albumin. *: p<0.05 (3-week-old Glast+/+ mouse vs retina of 3-week-old Glast−/− mouse). #: p<0.05 (3-week-old Glast+/+ mouse vs retina of 3-week-old Glast−/− mouse). FIG. 17C shows the rates of the fragmented or shrunken nuclei detected by the Hoechst staining in 24 hours after treatment with a control (C; HBSS), glutamate (Glu; 300 μM glutamate+10 μM glycine) or Glu+100 ng protein/mL E-LP in the absence or presence of □2 macroglobulin. *: p<0.05 (Glu+E-LP vs Glu+E-LP+a2M). FIG. 17D shows the state of RGC with Glu added after incubation for 15 minutes together with 100 nM a2M and E-LP (100-3,000 ng protein/mL). The data of FIGS. 17C and D were represented as average±SE values from the results of four experiments. * and **: p<0.05 and p<0.005 (each Glu+a2M+E-LP), respectively.

### Example 28

This example was to examine the amount of α2-macroglobulin released from the glia cells. To a culture mixture of the glia cells obtained by the primary culture, human lipoprotein containing apoE (100 or 1,000 ng/mL) was added, followed by collection of a culture supernatant after 24 hours. After the resulting supernatant was subjected to SDS-PAGE, it was detected by the immunoblotting using anti-α2-macroglobulin antibody. As a result, a decrease in the amount of α2-macroglobulin released from the glia cells was observed by the addition of the human lipoprotein containing apoE (FIG. 18).

It was considered from this result that the administration of the human lipoprotein containing apoE into the vitreous body may possess the directly neuroprotective effects as well as the effects for decreasing the amount of α2-macroglobulin released into the vitreous humor, which hinders the neuroprotective effects of the lipoprotein containing apoE.

### INDUSTRIAL APPLICABILITY

The present invention provides a prophylactic and therapeutic agent for neurological diseases and a prophylactic and therapeutic method for neurological diseases such as various neurodegenerative diseases accompanied by apoptosis of nerve cells due to the inevitable condition, which uses the lipoprotein containing apolipoprotein E having the neuroprotective action and effects such as the action and effects for inhibiting neurodegeneration or the neuroprotective system utilizing the activation of a neuroprotective molecule and the inactivation of a neurodegeneration-inducing molecule, through a mediation of the lipoprotein receptor. Therefore, the prophylactic and therapeutic agent of the present invention for neurological diseases and the prophylactic and therapeutic method of the present invention for neurological diseases are useful for a prevention and treatment of neurological diseases including such as neurodegenerative diseases.

1. An inhibitor of a neuroprotective inhibitory effect of α2-macroglobulin for inhibiting the neuroprotective inhibitory effect of α2-macroglobulin by administration onto the eyeball or injection into the eyeball thereof, comprising:
lipoprotein containing an apolipoprotein E.

2. An ophthalmologic composition containing a lipoprotein containing an apolipoprotein E, wherein:
an activity of a glutamic acid receptor of retinal ganglion cells present in the eyeball is inhibited by said lipoprotein containing apolipoprotein E by administration of the composition onto or into an eyeball by injection thereof, and the neuroprotective inhibitory effects of α2-macroglobulin is inhibited.

3. The ophthalmologic composition, as claimed in claim 2, wherein:
said lipoprotein containing apolipoprotein E is one of a group consisting of:
a lipoprotein containing a glia-cell-derived apolipoprotein E, a high density lipoprotein containing a lipoprotein containing apolipoprotein E separated from the blood or a reconstituted artificial lipoprotein containing apolipoprotein E.

4. The ophthalmologic composition; as claimed in claim 2, wherein:
said lipoprotein containing apolipoprotein E is a neuroprotective molecule exhibiting a neuroprotective effect by activation and a neurodegeneration-inducing molecule exhibiting a neuroprotective effect by inactivation or inhibition, through a mediation of a lipoprotein receptor.

5. The ophthalmologic composition, as claimed in claim 4, wherein:
said lipoprotein receptor is selected from a group consisting of:
LRP1 receptor, LDL receptor, ApoER2 receptor, VLDL receptor, LR11 receptor, LRP4 receptor, LRP1B receptor, Megalin receptor, LRP5 receptor and LRP6 receptor.

6. The ophthalmologic composition as claimed in claim 4, wherein:
said neuroprotective molecule is at least one of a phospholipase C and a protein kinase Cδ, and
said neurodegeneration-inducing molecule is at least any one of NMDA receptor, calcium and GSK3β.

7. (canceled)

8. The ophthalmologic composition as claimed in claim 2, characterized by inhibition of an apoptosis of said retinal ganglion cells.

9. A use of the ophthalmologic composition, as claimed in claim 2 as one of a prophylactic or therapeutic agent for diabetic retinopathy.
